(19) United States
(12) Patent Application Publication
(10) Pub. No.: US 2024/0149265 A1
(43) Pub. Date: May 9, 2024

(54) AUXILIARY TEST PAPER DETECTION DEVICE

(71) Applicant: JIANGSU BIOPERFECTUS TECHNOLOGIES CO., LTD, Taizhou, Jiangsu (CN)

(72) Inventors: Qianqian Shang, Taizhou, Jiangsu (CN); Qianqian JIA, Taizhou, Jiangsu (CN); Xinyi JIANG, Taizhou, Jiangsu (CN); Linping TAN, Taizhou, Jiangsu (CN); Xin WANG, Taizhou, Jiangsu (CN); Fancheng WEI, Taizhou, Jiangsu (CN); Renyuan CHEN, Taizhou, Jiangsu (CN); Wei JIN, Taizhou, Jiangsu (CN); Zhonghua LIU, Taizhou, Jiangsu (CN); Guoqiang WANG, Taizhou, Jiangsu (CN)

(73) Assignee: JIANGSU BIOPERFECTUS TECHNOLOGIES CO., LTD., Taizhou, Jiangsu, JS (CN)

(21) Appl. No.: 17/778,693

(22) PCT Filed: Sep. 26, 2021
(86) PCT No.: PCT/CN2021/120553

§ 371 Date: May 20, 2022

(30) Foreign Application Priority Data
Mar. 25, 2021 (CN) 202110319458.3
Jul. 28, 2021 (CN) 202110859547.7

(51) Int. Cl. B01L 3/00; B01L 9/00

(52) U.S. Cl. CPC B01L 3/5023; B01L 9/52; B01L 2200/026; B01L 2200/0689; B01L 2200/16; B01L 2300/025; B01L 2300/042; B01L 2300/069

(57) ABSTRACT

An auxiliary test paper detection device includes a reagent cap and a test paper cassette. The reagent cap contains a detection reagent, a sealing member is arranged at an opening of the reagent cap. The sealing member can be opened or removed under action of external force. Test paper is arranged in the test paper cassette, a sample absorption section of the test paper is exposed, an observation window is formed in the test paper cassette. A result display area of the test paper corresponds to the observation window. A connecting structure for connecting the test paper cassette and the reagent cap is provided, and when the test paper cassette and the reagent cap are connected through the connecting structure, a closed space is formed in an inner cavity of the reagent cap. The sample absorption section of the test paper extends into the inner cavity of the reagent cap.

### TECHNICAL FIELD

The present disclosure relates to the technical field of rapid diagnosis detection, and more specifically, relates to an auxiliary test paper detection device.

### BACKGROUND ART

Medical diagnosis of epidemics usually requires etiology-related examinations in laboratories, mainly including virus isolation, viral antigen, nucleic acid and antibody detection, and the like. The virus isolation is the “gold standard” for laboratory detection.

Virus isolation culture is to separate influenza viruses from respiratory tract specimens, but generally, it takes long time, has a high n culture difficulty and a low success rate. Viral antigen detection is a rapid detection method capable of adopting an immunofluorescence to detect mucosal epithelial cells in respiratory tract specimens (pharyngeal swabs, nasal swabs, nasopharyngeal swab or tracheal extracts), results can be generally obtained within 10-30 minutes, and the viral antigen detection is simple to operate and easy to use. Serological diagnosis is that the IgG antibody level by dynamic detection is increased by four times or above in the recovery period compared with that in the acute period, retrospective diagnostic significance is achieved, but double samples need to be collected for comparison. Virus nucleic acid detection has the best specificity and sensitivity, can quickly distinguish virus types and subtypes, can generally obtain results within 4-6 hours, but has high requirements for laboratory equipment and operator ability. Antibody detection can be used for retrospective investigation, but is not significant for early diagnosis of cases.

Comprehensively, existing viral antigen detection methods are easier to be recognized and accepted by the general public, pathogen detection needs to be carried out in hospital laboratories at present, but some problems exist in hospital treatment: specifically, firstly, the number of patients is large, and the waiting time of procedures such as queuing registration, waiting for diagnosis, examination, medicine taking and treatment is long and complex; secondly, the possibility of cross-infection between the patients is also caused by the contact of a plurality of patients in hospital; and thirdly, the economic burden is relatively high.

Therefore, a pathogen detection device which is safe and simple to operate and accurate in detection result is needed.

In addition, in current medical diagnosis products, detection of various to-be-detected sample (nasopharyngeal secretions, saliva, urine, blood, sweat and the like) usually requires pretreatment of the samples. Generally, a sample to be detected is pretreated (diluted or eluted) through an independent sample treatment device filled with a required buffer solution, so that a sample more suitable for detection is obtained. The sample is manually transferred from the sample treatment device into a sample adding hole provided with a test paper detection device, and after the reaction time is finished, a detection result is observed by naked eyes or read by virtue of an instrument. Whether the sample contains the detected target object or not is determined according to the detection result. Said method is a relatively mature and general method at present. However, in the detection process, the sample is transferred from the sample treatment device to the detection device, and multiple operation steps are needed, so that the detection process is complicated. In addition, generally, a pipettor or a plastic straight suction pipe is used for transferring the sample, so that the biological pollution of the environment is caused because the sample accidentally drops or excessive sample overflows from the sample adding hole due to the fact that dropping of the sample is not controlled in a detection area in the sample transferring process; and an inaccurate detection result may be caused by inaccurate transferred sample volume due to non-standard operation of an operator using the pipettor or the plastic straight pipette. At present, pathogen detection needs to be carried out in hospital laboratories at present, but a series of problems exist in hospital treatment: specifically, firstly, the number of patients is large, and the waiting time of procedures such as queuing registration, waiting for diagnosis, examination, medicine taking and treatment is long and complex; secondly, the possibility of cross-infection between the patients is also caused by the contact of a plurality of patients in hospital; and thirdly, the economic burden is relatively high.

Therefore, a detection device which can avoid environmental biological pollution and manual sample adding errors and is convenient to operate autonomously at home is also needed.

### SUMMARY

Aiming at the shortages existing in the prior art, the present disclosure aims to provide an auxiliary test paper detection device.

The above technical purpose of the present disclosure is realized through the following technical scheme.

It is provided an auxiliary test paper detection device. The auxiliary test paper detection device includes a reagent cap and a test paper cassette. The reagent cap is configured for containing a detection reagent, a sealing member is arranged at an opening of the reagent cap, and the sealing member is opened or removed under action of external force; test paper is arranged in the test paper cassette, a sample absorption section of the test paper is exposed, an observation window is formed in the test paper cassette, and a result display area of the test paper corresponds to the observation window. A connecting structure for connecting the test paper cassette and the reagent cap is provided; when the test paper cassette and the reagent cap are connected through the connecting structure, a closed space is formed by an inner cavity of the reagent cap, and the sample absorption section of the test paper extends into the inner cavity of the reagent cap.

In some embodiments, the test paper cassette includes an upper cover and a lower base which are in snap fit with each other, a gap for accommodating the test paper is formed between the upper cover and the lower base, and the upper cover and the lower base each are provided with positioning fasteners and first positioning blocking ribs. When the upper cover and the lower base are in snap fit, the positioning fasteners on the upper cover and the lower base are in snap fit with each other to form positioning lock, and the first positioning blocking ribs on the upper cover and the lower base cooperatively and tightly abut against the test paper.

In some embodiments, the connecting structure includes a connecting clamping sleeve and tail end fasteners, an inner periphery of an inner ring of the connecting clamping sleeve is fixedly sleeved on the test paper cassette, and an outer periphery of the inner ring of the connecting clamping sleeve is sleeved with the reagent cap and in interference fit with the reagent cap. The tail end fasteners are elastic, and the tail end fasteners are respectively arranged on the upper cover and the lower bottom at an end of the test paper cassette, fixedly sleeved with the connecting clamping sleeve. When the connecting clamping sleeve is sleeved to a predetermined station, the inner ring of the connecting clamping sleeve is in snap fit with the tail end fasteners on the upper cover and the lower base.

In some embodiments, the test paper cassette includes an upper cover and a lower base, a positioning hole is formed in the upper cover, a positioning convex block is arranged on the lower base, the positioning convex block is embedded in the positioning hole and in interference fit with the positioning holes; a gap for accommodating the test paper is formed between the upper cover and the lower base; and an end of the test paper cassette, from which the sample absorption section of the test paper extends out, has a shape adapted to an inner wall of the reagent cap.

In some embodiments, the connecting structure includes two handles, the two handles are respectively and movably arranged at two sides of the end of the test paper cassette, from which the sample absorption section of the test paper extends out, and main bodies of the two handles extend outwards in an insertion direction of the reagent cap.

In some embodiments, the test paper cassette includes an upper cover and a lower base which are in snap fit with each other, a gap for accommodating the test paper is formed between the upper cover and the lower base, positioning mounting holes are formed in the upper cover, and mounting columns are arranged on the lower base. When the upper cover and the lower base are in snap fit with each other, the mounting columns are inserted into the positioning mounting holes and in interference fit with the positioning mounting holes.

In some embodiments, the connecting structure includes an insertion member, an outer contour of the insertion member corresponds to a side wall of an inner cavity of the reagent cap, and the insertion member is in interference fit with the side wall of the inner cavity of the reagent cap.

In some embodiments, the test paper cassette includes an upper cover and a lower base which are in snap fit with each other, a gap for accommodating the test paper is formed between the upper cover and the lower base, positioning fasteners are arranged on the upper cover and the lower base, and second positioning blocking ribs are arranged on the upper cover and the lower base. When the upper cover is in snap fit with the lower base, the positioning fasteners on the upper cover and the lower base are in snap fit together and form a positioning lock, and the second positioning blocking ribs on the upper cover and the lower base cooperatively and tightly abut against the test paper.

In some embodiments, the connecting structure includes an elastic sealing member, the elastic scaling member is arranged between the test paper and the test paper cassette, and the elastic scaling member is located at an end of the test pater cassette where the sample absorption section of the test paper is located. The end of the test pater cassette where the sample absorption section of the test paper is located has a shape adapted to an outer wall of the reagent cap, and an outer contour of the elastic sealing member is adapted to the inner cavity of the reagent cap.

In some embodiments, the elastic sealing member includes inner rubber plugs and an outer rubber plug, the inner rubber plugs include a pair of split rubber plugs which are connected with each other in an insertion manner, the inner rubber plugs are sleeved on the test paper, one end of the outer rubber plug is sleeved on the inner rubber plugs to form secondary sealing, and another end of the outer rubber plug is inserted into the inner cavity of the reagent cap to form sealing.

It is further provided an auxiliary detection device configured for auxiliary detection of a test paper bearing sample and display of a detection result, including a reagent bearing device and a test paper cassette;
the reagent bearing device is a container provided with a sealing member at an opening of the container and configured for containing a certain amount of detection reagent and bearing sample solution during detection, and the sealing member is opened or removed under action of external force;
the test paper cassette is configured for positioning and assembling test paper and clamping the reagent bearing device, when the test paper and the test paper cassette are assembled, a sample absorption section of the test paper is exposed outside the test paper cassette, and a result display area of the test paper is arranged in the test paper cassette and just corresponds to a window of the test paper cassette; and when the test paper cassette clamps the reagent bearing device, the sample absorption section of the test paper is immersed into the sample solution of the reagent bearing device to implement sample bearing.

The test paper is assembled in the lower bottom according to an upper alignment mode; the upper cover and the lower bottom are sealed. The clamping sleeve clamps on the convex parts of the upper cover and the lower bottom, from bottom to top, until the clamping sleeve not pushed to perform a press fit to form a combined body. After a certain amount of liquid is filled in the reagent bearing device in advance, the reagent bearing device is packaged individually so as to be convenient for household use, which avoids the step of manually transferring samples and reduces inaccurate detection results caused by inaccurate sample transfer amount. Furthermore, the reagent bearing device is sealed so as to avoid overflow of the liquid in transportation. During use, a sealing member of the reagent bearing device is opened, the sample absorption section of the test paper is vertically inserted into the reagent bearing device filled with the liquid, and the reagent bearing device is inserted into the cassette until the reagent bearing device is not pushed, so that the reagent bearing device is completely scaled, thereby preventing the detection environment from pollution caused by liquid overflow; and the detection device is horizontally placed on a table, after timing reaction is carried out for 10-15 minutes, and the detection result can be obtained by inserting an adapted instrument or observing through naked eyes.

In some embodiments, the test paper cassette includes an upper cover and a lower base which are in snap fit with each other, at least the result display area of the test paper is arranged between the upper cover and the lower base which are in snap fit together, and a display window is formed in the upper cover or the lower base.

In some embodiments, the auxiliary detection device further includes a clamping sleeve, where an inner periphery of an inner ring of the clamping sleeve is sleeved on tail portions of the upper cover and the lower base, and an outer periphery of the inner ring of the clamping sleeve is sleeved with the reagent bearing device.

In some embodiments, working surfaces of the upper cover and the lower base are respectively and sequentially provided with end positioning fasteners, test paper accommodating grooves, positioning blocking ribs and tail end fasteners.

When the upper cover and the lower base are in snap fit together, the end positioning fasteners of the upper cover and the lower base are in snap fit with each other to form positioning lock.

The test paper accommodating grooves of the upper cover and the lower base are matched with each other to form an accommodating space for placing the test paper.

The positioning blocking ribs of the upper cover and the lower base are matched with each other to form a limiting member for preventing the test paper from sliding into and out of the test paper cassette.

Tail end fasteners of the upper cover and the lower base are assembled together to form a fastener which has a shape adapted to the inner ring of the clamping sleeve, and when the clamping sleeve is sleeved to a predetermined station, the inner ring of the clamping sleeve abuts against and is fixed with the tail end fasteners on the upper cover and on the lower base.

In some embodiments, a large-to-small structure of an inner ring of the reagent bearing device is in interference fit with a small-to-large structure of the inner ring of the clamping sleeve, to allow liquid in the reagent bearing device to not leak.

It is provided an auxiliary detection device configured for auxiliary detection of a test paper bearing sample and display of a detection result, the auxiliary detection device includes a reagent bearing device and a test paper cassette.

The reagent bearing device is a container provided with a sealing member at an opening of the container and configured for containing a certain amount of detection reagent and bearing sample solution during detection, and the sealing member is opened or removed under action of external force.

The test paper cassette is configured for positioning and assembling test paper and being sleeved on the reagent bearing device. When the test paper and the test paper cassette are assembled, a sample absorption section of the test paper is exposed outside the test paper cassette. A tail portion of the test paper cassette wraps the test paper to form local sealing, and a result display area of the test paper is arranged in the test paper cassette and just corresponds to a window of the test paper cassette. When the test paper cassette is inserted into the reagent bearing device, the tail portion of the test paper cassette and the inner cavity of the reagent bearing device form a closed space, and the sample absorption section of the test paper is immersed into the sample solution of the reagent bearing device to implement sample bearing.

In some embodiments, the test paper cassette includes a cover for clamping test paper. The sample absorption section of the test paper extends out from a tail portion of the cover, the tail portion of the cover has a shape adapted to the inner cavity of the reagent bearing device. A pair of handles are arranged at two sides of the tail portion of the cover. When the reagent bearing device with the sealing member removed is inserted into the tail portion of the cover, the pair of handles just abut against two side walls of the reagent bearing device.

In some embodiments, the handles are arranged at a tail portion of the lower base, one end of each handle is connected with a side wall of the lower base to form one piece, and main bodies of the handles extend outwards along an insertion direction of the reagent bearing device.

In some embodiments, an arc-shaped groove for fingers to grasp is formed in an outer side of each handle.

An auxiliary detection device configured for auxiliary detection of a test paper bearing sample and display of a detection result is provided by the present disclosure. The auxiliary detection device includes a test paper cassette and a reagent bearing device. The test paper cassette includes a cover for clamping test paper, a sample absorption section of the test paper extends out from a tail portion of the cover. The tail portion of the cover has a shape adapted to an inner cavity of the reagent bearing device. The reagent bearing device is a container provided with a sealing member at an opening of the container and configured for containing a certain amount of detection reagent and bearing sample solution during detection, and the sealing member is opened or removed under action of external force.

In some embodiments, the cover is configured for positioning and assembling the test paper and being sleeved on the reagent bearing device. The test paper and the cover are assembled, the sample absorption section of the test paper is exposed outside the test paper cassette, a tail portion of the test paper cassette wraps the test paper to form local partition. A result display area of the test paper is arranged in the test paper cassette and just corresponds to a window of the test paper cassette. When the test paper cassette is inserted into the reagent bearing device, the tail portion of the test paper cassette and the inner cavity of the reagent bearing device form a closed space, and the sample absorption section of the test paper is immersed into the sample solution of the reagent bearing device to implement sample bearing.

In some embodiments, contacting surfaces of the upper cover and the lower base are respectively and sequentially provided with end positioning fasteners, test paper accommodating grooves, tail positioning fasteners and tail end insertion heads.

When the upper cover and the lower base are in snap fit, the positioning fasteners of the upper cover and the lower base are in snap fit with each other to form positioning lock.

The test paper accommodating grooves of the upper cover and the lower base are matched with each other to form an accommodating space for placing the test paper.

Tail ends of the upper cover and the lower base are assembled together to form an insertion member which has a shape adapted to the inner ring of the inner cavity of the reagent bearing device, and the insertion member is in interference fit with the inner cavity of the reagent bearing device.

In some embodiments, the test paper accommodating groove of the lower base is internally provided with a plurality of supporting columns used for supporting the test paper.

An auxiliary detection device configured for auxiliary detection of a test paper bearing sample and display of a detection result is provided by the present disclosure. The auxiliary detection device includes a test paper cassette and a reagent bearing device. The test paper cassette includes a cover for clamping test paper. A sample absorption section of the test paper is erected in a space in a tail portion of the cover, a sealing member is filled between the tail portion of the cover and the test paper. The tail portion of the cover has a shape adapted to an outer cavity of the reagent bearing device, and an outer contour of the sealing member is adapted to an inner cavity of the reagent bearing device. The reagent bearing device is a container provided with a sealing member at an opening of the container and configured for containing a certain amount of detection reagent and bearing sample solution during detection, and the sealing member is opened or removed under action of external force.

The cover is used for positioning and assembling the test paper and being sleeved on the reagent bearing device. When the test paper and the cover are assembled, the sample absorption section of the test paper is erected in a space in a tail portion of the test paper cassette. The test paper is wrapped with the sealing member, a local partition is formed between the tail portion of the test paper cassette and the test paper, and a result display area of the test paper is arranged in the test paper cassette and just corresponds to a window of the test paper cassette. When the test paper cassette is inserted into the reagent bearing device, the sealing member and the inner cavity of the reagent bearing device form a closed space. The sample absorption section of the test paper is immersed into sample solution of the reagent bearing device to complete sample bearing work.

In some embodiments, the sealing member includes inner rubber plugs and an outer rubber plug. The inner rubber plugs include a pair of split rubber plugs which are connected with each other in an insertion manner. The pair of inner rubber plugs are sleeved on a predetermined position of the test paper, the inner rubber plugs which are connected are inserted into one end of the outer rubber plug to form secondary sealing, and another end of the outer rubber plug is inserted into the inner cavity of the reagent bearing device to form sealing.

In some embodiments, the test paper cassette includes an upper cover and a lower base which are snap fit with each other, at least the result display area of the test paper is clamped between the upper cover and the lower base, and a display window is formed in the upper cover or the lower base.

In some embodiments, working surfaces of the upper cover and the lower base are respectively and sequentially provided with end positioning fasteners, test paper accommodating grooves, positioning blocking ribs and tail end fasteners. When the upper cover and the lower base are in snap fit with each other, the end positioning fasteners of the upper cover and the lower base are in snap fit with each other to form positioning lock. The test paper accommodating grooves of the upper cover and the lower base are matched with each other to form an accommodating space for placing the test paper. The positioning blocking ribs of the upper cover and the lower base are matched with each other to form a limiting member for preventing the test paper to sliding in and out of the test paper cassette.

In some embodiments, the sealing member is a sealing film or other covers with sealing effect.

The present disclosure aims to solve the problem that people can conveniently perform pathogen detection in families after influenza A test strip and influenza B test strip are combined, whether body discomfort is caused by influenza virus or not can be preliminarily judged, and whether the body discomfort is caused by influenza A virus or influenza B virus can be further distinguished.

Compared with the prior art, the present disclosure has the following beneficial effects.

Firstly, liquid is sub-packaged in the reagent cap in advance, and the test paper cassette and the reagent cap are connected in an insertion mode, which is simple in operation and facilitates to reduce manual operation and result errors caused by manual sample adding.

Secondly, a closed reaction device is formed in the reagent cap, so that biological pollution is reduced when the present disclosure is used in a household environment, thereby protecting the user and the household environment.

Thirdly, by self-testing pathogens at home, cross infection caused in hospitals is avoided, and the waste of manpower, material resources and financial resources caused by queuing and registering in hospitals is avoided;

Fourthly, different types of influenza pathogens can be detected by using different test paper, and other similar respiratory pathogens can also be detected, so that the applicability of the auxiliary test paper detection device can be improved.

Fifthly, the whole detection device is a closed reaction device, so that biological pollution is reduced when the detection device is used in a household environment, thereby protecting the user and the household environment.

Sixthly, by self-testing pathogens at home, cross infection caused in hospitals is avoided, and the waste of manpower, material resources and financial resources caused by queuing and registering in hospitals is avoided;

The detection device disclosed by the present disclosure is wide in adaptability, and can be matched with test paper of other items for testing (such as other respiratory pathogens, hormones and the like), so that not only influenza pathogens can be detected at home, but also other similar respiratory pathogens, cancer targets and hormone targets can be detected, thereby providing further convenience to people.

### BRIEF DESCRIPTION OF THE DRAWINGS

To describe the technical scheme in the embodiments of the present disclosure or in the prior art more clearly, the following briefly introduces the accompanying drawings required for describing the embodiments. Apparently, the accompanying drawings in the following description show merely some embodiments of the present disclosure, and those skilled in the art may still derive other accompanying drawings from these accompanying drawings without creative efforts.

FIG. 1 is a cross-sectional schematic diagram of an overall structure of an auxiliary test paper detection device in Embodiment 1 of the present disclosure;

FIG. 2 is an external schematic diagram of the overall structure of the auxiliary test paper detection device in Embodiment 1 of the present disclosure;

FIG. 3 is an explosive schematic diagram of the overall structure of the auxiliary test paper detection device in Embodiment 1 of the present disclosure;

FIG. 4 is a schematic diagram of an overall structure of a reagent cap in Embodiment 1 of the present disclosure;

FIG. 5 is a cross-sectional schematic diagram of an overall structure of an auxiliary test paper detection device in Embodiment 2 of the present disclosure;

FIG. 6 is an external schematic diagram of the overall structure of the auxiliary test paper detection device in Embodiment 2 of the present disclosure;

FIG. 7 is an explosive schematic diagram of the overall structure of the auxiliary test paper detection device in Embodiment 2 of the present disclosure;

FIG. 8 is a cross-sectional schematic diagram of an overall structure of an auxiliary test paper detection device in Embodiment 3 of the present disclosure;

FIG. 9 is an external schematic diagram of the overall structure of the auxiliary test paper detection device in Embodiment 3 of the present disclosure;

FIG. 10 is an explosive schematic diagram of the overall structure of the auxiliary test paper detection device in Embodiment 3 of the present disclosure;

FIG. 11 is a cross-sectional schematic diagram of an overall structure of an auxiliary test paper detection device in Embodiment 4 of the present disclosure; and

FIG. 12 is an explosive schematic diagram of the overall structure of the auxiliary test paper detection device in Embodiment 4 of the present disclosure.

List of Reference signs: 1 lower base; 2′ upper cover; 21′ ventilated window; 22′ observation window; 3′ test paper; 4′ reagent cap: 5′ positioning fastener; 51′ first positioning blocking rib; 52′ connecting clamping sleeve; 53′ tail end fastener; 6 positioning hole; 61′ positioning convex block; 62′ handle: 7′ positioning mounting hole; 71′ mounting column; 72′ insertion member; 8′ positioning fastener; 81′ second positioning blocking rib; 82′ lower inner rubber plug; 83′ upper inner rubber plug; and 84′ outer rubber plug.

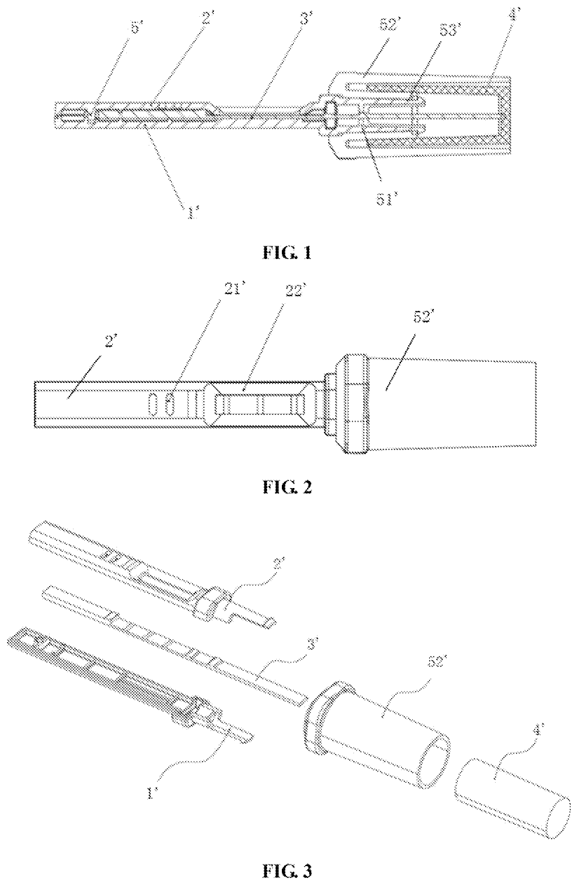

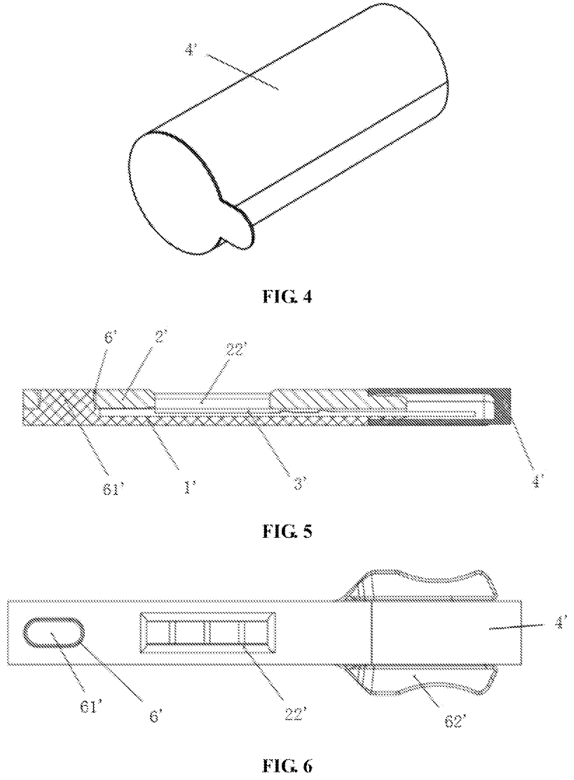

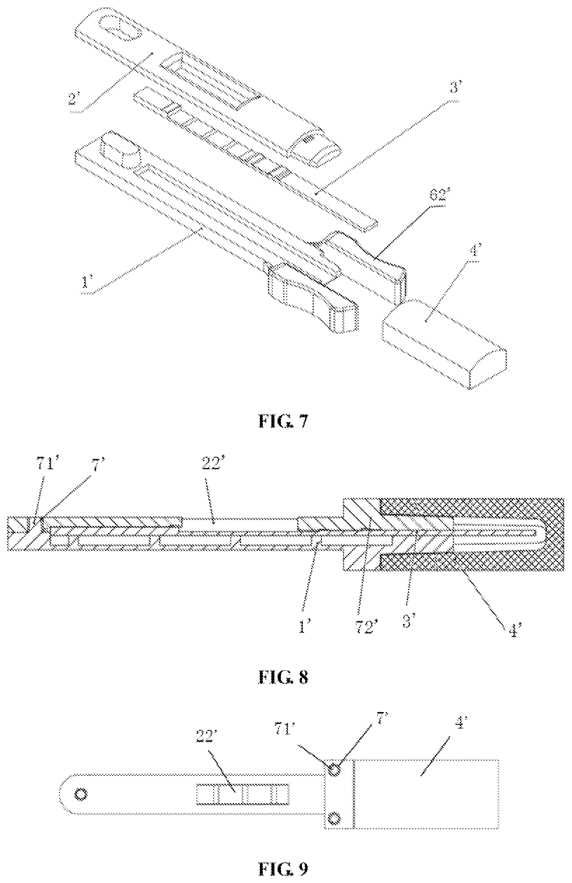

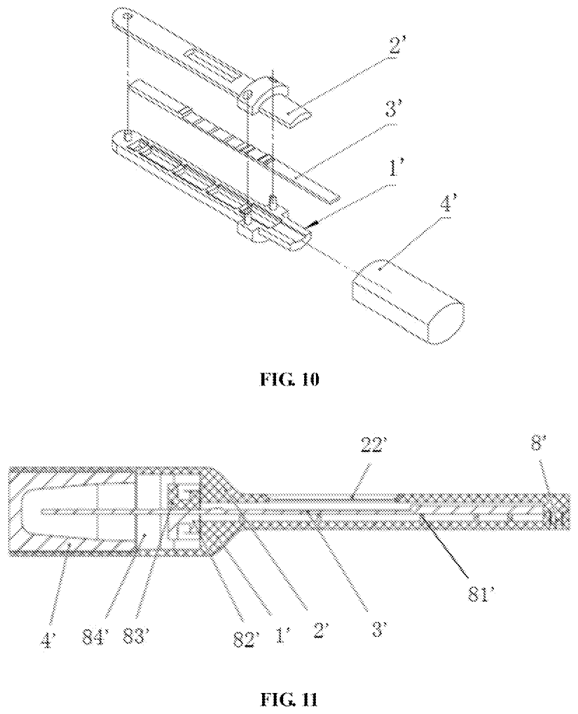

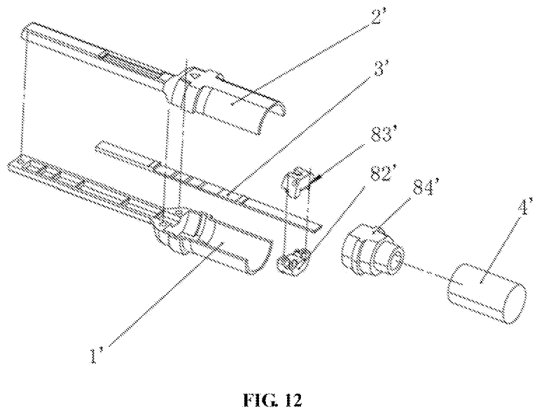

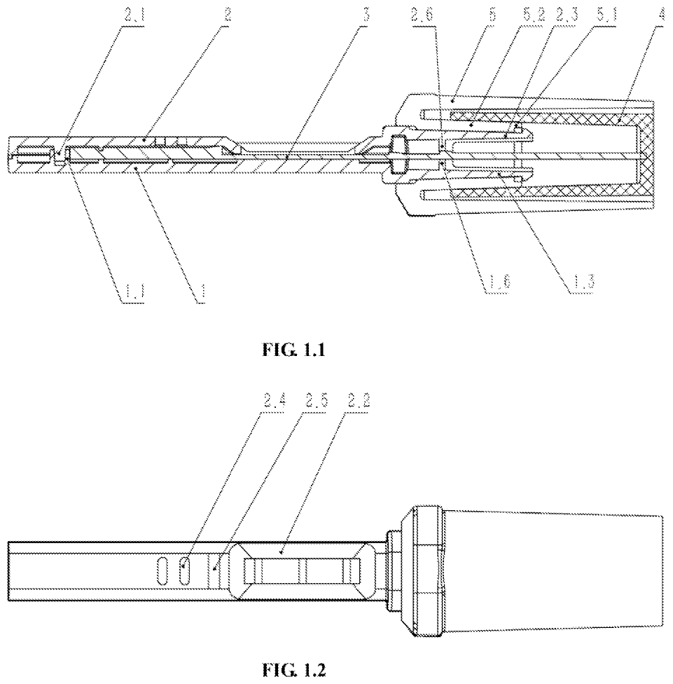

FIG. 1.1 is a cross-sectional structural schematic diagram of Embodiment 5:

FIG. 1.2 is a structural schematic diagram in top view of Embodiment 5:

FIG. 1.3 is an assembly structural schematic diagram of Embodiment 5; and

FIG. 1.4 is a stereoscopic schematic diagram of a reagent bearing device in Embodiment 5.

List of reference signs: 1 lower base; 1.1 first positioning fastener, 1.3 lower base tail end fastener, 1.6 lower base positioning blocking rib; 2 upper cover; 2.1 second positioning fastener, 2.3 upper cover tail end fastener: 2.2 reaction window; 2.6 upper cover positioning blocking rib; 3 test paper; 4 reagent bearing device; 5 clamping sleeve; 5.1 clamping-sleeve inner ring tail end; and 5.2 clamping-sleeve inner ring middle section.

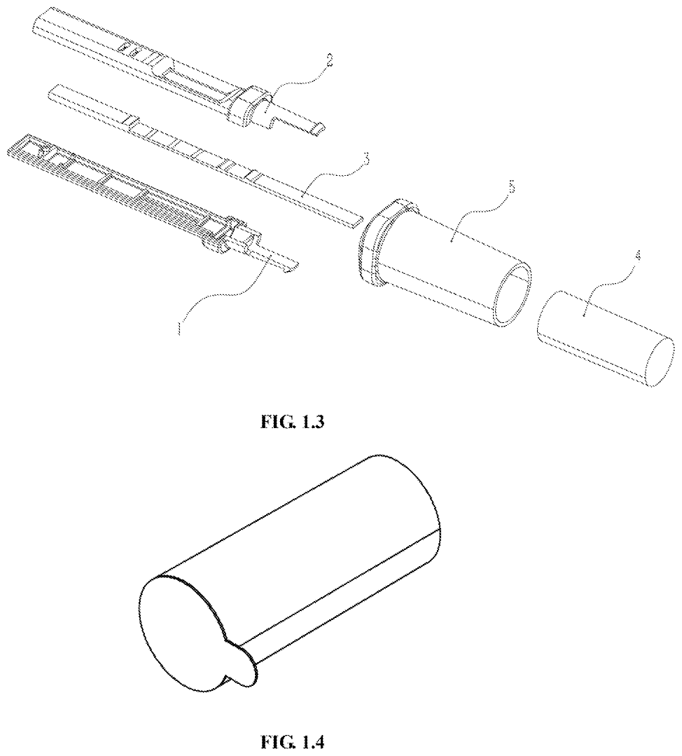

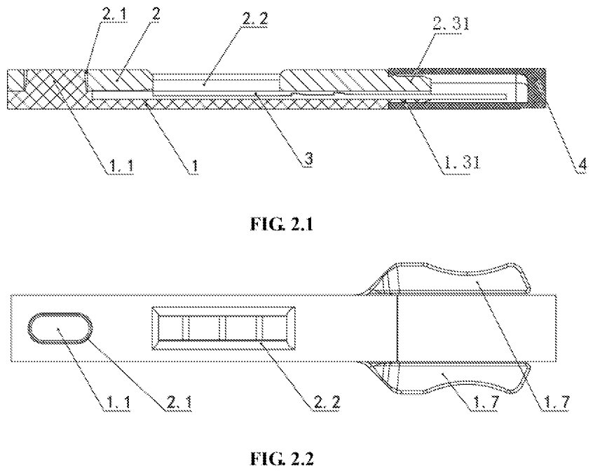

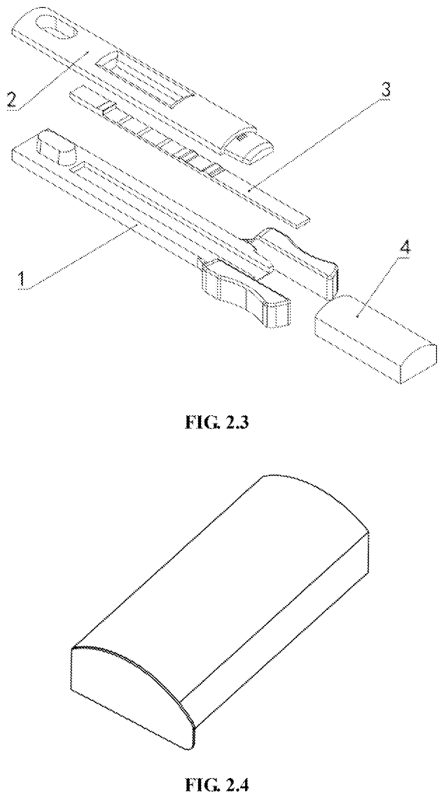

FIG. 2.1 is a cross-sectional structural schematic diagram of Embodiment 6;

FIG. 2.2 is a structural schematic diagram in top view of Embodiment 6;

FIG. 2.3 is an assembly structural schematic diagram of Embodiment 6; and

FIG. 2.4 is a stereoscopic schematic diagram of a reagent bearing device in Embodiment 6.

List of reference signs: 1 lower base; 1.1 first positioning fastener; 1.31 lower base tail end; 1.7 lower base handle; 2 upper cover; 2.1 second positioning fastener; 2.31 upper cover tail end; 2.2 reaction window; 3 test paper; and 4 reagent bearing device.

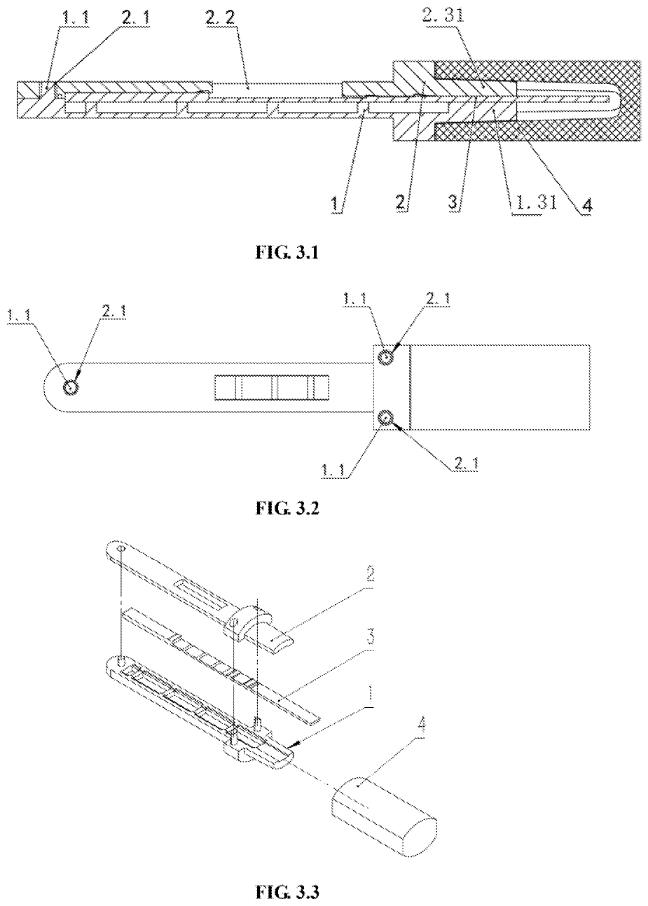

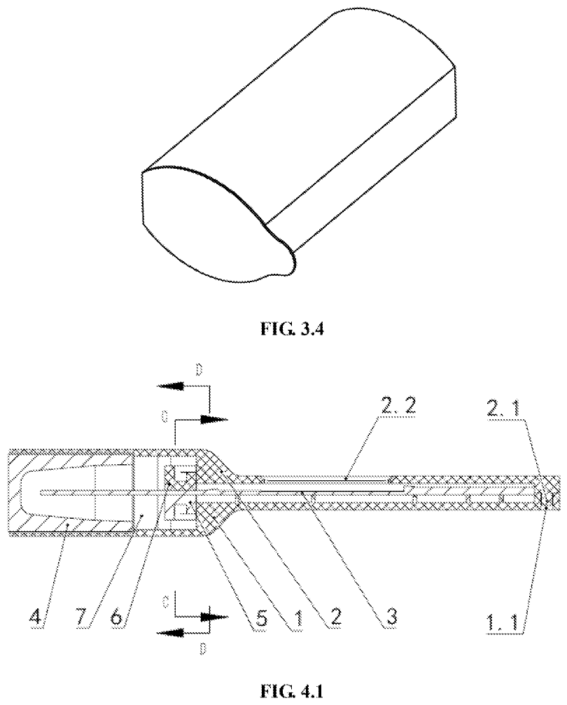

FIG. 3.1 is a cross-sectional structural schematic diagram of Embodiment 7;

FIG. 3.2 is a structural schematic diagram in top view of Embodiment 7:

FIG. 3.3 is an assembly structural schematic diagram of Embodiment 7; and

FIG. 3.4 is a stereoscopic schematic diagram of a reagent bearing device in Embodiment 7.

List of reference signs: 1 lower base; 1.1 first positioning fastener; 1.31 lower base tail end; 2 upper cover; 2.1 second positioning fastener; 2.2 reaction window; 2.31 upper cover tail end; 3 test paper; and 4 reagent bearing device.

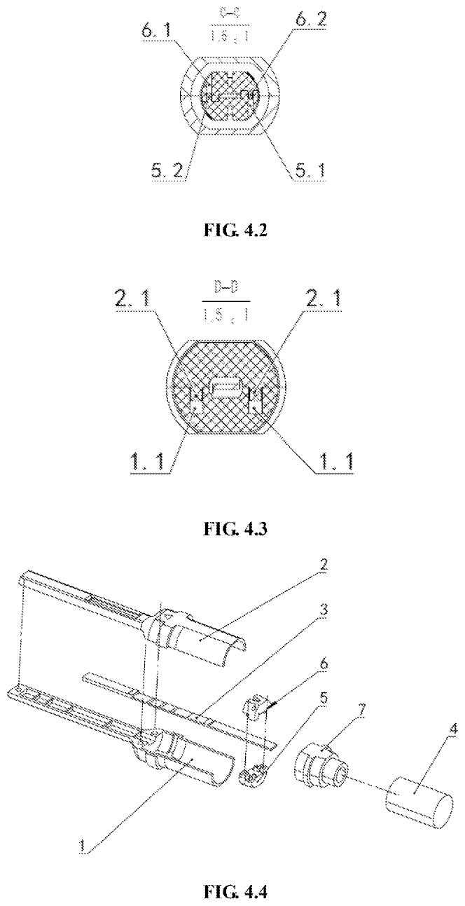

FIG. 4.1 is a cross-sectional structural schematic diagram of Embodiment 8;

FIG. 4.2 is a cross-sectional structural schematic diagram along the C-C direction in FIG. 4.1;

FIG. 4.3 is a cross-sectional structural schematic diagram along the D-D direction in FIG. 4.2;

FIG. 4.4 is an assembly structural schematic diagram of Embodiment 8; and

FIG. 4.5 is a stereoscopic schematic diagram of a reagent bearing device in Embodiment 8.

List of reference signs: 1 lower base; 1.1 first positioning fastener: 2 upper cover; 2.1 second positioning fastener; 2.2 reaction window; 3 test paper; 4 reagent bearing device; 51 lower inner rubber plug; 5.1 lower-inner-rubber-plug groove; 5.2 lower-inner-rubber-plug rivet; 6 upper inner rubber plug; 6.1 upper-inner-rubber-plug groove; 6.2 upper-inner-rubber-plug rivet; and 7 outer rubber plug.

### DETAILED DESCRIPTION OF THE EMBODIMENTS

The following clearly and completely describes the technical scheme in the embodiments of the present disclosure with reference to the accompanying drawings in the embodiments of the present disclosure. Apparently, the described embodiments are merely a part rather than all of the embodiments of the present disclosure. Based on the embodiments in the present disclosure, all other embodiments obtained by the ordinary technical staff in the art under the premise of without contributing creative labor belong to the scope protected by the present disclosure.

The present disclosure aims to provide an auxiliary test paper detection device to solve the problems in the prior art.

To make the foregoing objective, features and advantages of the present disclosure clearer and more comprehensible, the present disclosure is further described in detail below with reference to the accompanying drawings and specific embodiments.

### Embodiment 1

As shown in FIG. 1 to FIG. 3, the present disclosure provides an auxiliary test paper detection device. The auxiliary test paper detection device includes a reagent cap 4′ and a test paper cassette. The reagent cap 4′ is used for containing a detection reagent. Test paper 3′ is arranged in the test paper cassette, a sample absorption section of the test paper 3′ extends out from one end of the test paper cassette and is exposed, an observation window 22′ is formed in the test paper cassette, and a result display area of the test paper 3′ corresponds to the observation window 22′. A connecting structure is connected between the test paper cassette and the reagent cap 4′, and when the test paper cassette and the reagent cap 4′ are connected through the connecting structure, a closed space is formed in an inner cavity of the reagent cap 4′, and the sample absorption section of the test paper 3′ extends into the inner cavity of the reagent cap 4′.

The test paper cassette includes an upper cover 2′ and a lower base 1′ which are in snap fit with each other, the upper cover 2′ and the lower base 1′ are both of strip structures, a gap for accommodating test paper 3′ is formed between the upper cover 2′ and the lower base 1′. The test paper 3′ is fixed through snap fit of the upper cover 2′ and the lower base 1′. An observation window 22′ is formed in the upper cover 2′, a result display area of the test paper 3′ corresponds to the observation window 22′, and the sample absorption section of the test paper 3′ extends out of the length end of the test paper cassette.

Positioning fasteners 5′ and first positioning blocking ribs 51′ are integrally formed on the upper cover 2′ and the lower base 1′. The upper cover 2′ and the lower base 1′ are assembled into one piece through the two positioning fasteners 5′, and the two positioning fasteners 5′ are located on the end of the test paper cassette away from the sample absorption section of the test paper 3′.

A plurality of first positioning blocking ribs 51′ are integrally formed in the middle of the upper cover 2′ and the middle of the lower base 1′ at intervals, each first positioning blocking rib 51′ abuts against the test paper 3′, the test paper 3′ is clamped and fixed through the first positioning blocking ribs 51′ so as to prevent the test paper 3′ from sliding inwards and outwards. When the upper cover 2′ and the lower base 1′ are in snap fit, interference fit is formed through the two positioning fasteners 5′, so that the upper cover 2′ and the lower base 1′ are completely sealed.

The connecting structure includes a connecting clamping sleeve 52′ and tail end fasteners 53′, the tail end fasteners 53′ are elastic, and one tail end fasteners 53′ are integrally formed respectively on the upper cover 2′ and the lower base 1′ at the end of the test paper cassette from which the sample absorption section of the test paper 3′ extends out. Inverted hook structures are formed at the ends of the two tail end fasteners 53′ away from the test paper cassette. The connecting clamping sleeve 52′ is of a hollow barrel shape, the inner diameter of the connecting clamping sleeve 52′ is gradually increased from one end to the other end, an inner ring is integrally formed in the connecting clamping sleeve 52′, the inner ring is fixedly connected with the end, with the largest inner diameter, of the connecting clamping sleeve 52′, and the inner ring and the inner wall of the connecting clamping sleeve 52′ are arranged at intervals.

The connecting clamping sleeve 52′ is sleeved with the test paper cassette from the end, with the sample absorption section of the test paper 3′, of the test paper cassette until the test paper cassette 52′ cannot be pushed, and through the cooperation of the two tail end fasteners 53′ and the inner ring of the connecting clamping sleeve 52′, when the connecting clamping sleeve 52′ is sleeved to a predetermined station, the inner ring of the connecting clamping sleeve 52′ respectively abuts against the inverted hook structures of the two tail end buckles 53′, so that it is guaranteed that the upper cover 2′ and the lower base 1′ are completely sealed.

As shown in FIG. 4′, the reagent cap 4′ is filled with the detection reagent, and the reagent cap 4′ is sealed with a sealing film or sealed by a cap, so that it is guaranteed that liquid in the device does not leak before use.

As shown in FIG. 1 and FIG. 3′, when detection is needed, a sealing member of the reagent cap 4′ is firstly opened, the test paper cassette is vertically inserted into the reagent cap 4′ with the sample absorption section of the test paper 3′ downwards until the test paper cassette is not pushed. The reagent cap and the connecting clamping sleeve are in interference fit through cooperation of the large-to-small structure of the inner ring of the reagent cap 4′ and the small-to-large structure of a middle section of the inner ring of the connecting clamping sleeve, which ensures that when the reagent cap 4′ is horizontally placed, liquid in the reagent cap 4′ cannot leak laterally. In addition, the sample absorption section of the test paper 3′ can be immersed into the liquid in the reagent cap 4′, the test paper 3′ is tightly clamped through the two first positioning blocking ribs 51′, the test paper 3′ can be firmly fixed; meanwhile, the liquid in the reagent cap 4′ is prevented from rushing to the test paper 3′ at the moment of horizontal placement. In 10-15 minutes of a chromatographic reaction, the liquid in the reagent cap 4′ can be gradually chromatographed from the sample absorption end of the test paper 3′ to the result display area of the test paper 3′ and then to the top end of the test paper 3′ until the chromatographic reaction is finished. Finally, a value is read by inserting the device into the instrument or by observing the observation window 22′ through the naked eyes.

As shown in FIG. 1 to FIG. 3, a ventilated window 21′ is further formed in the upper cover 2′, after 10-15 minutes of the reaction is finished, the instrument obtains a signal value through scanning and interpreting the area of the observation window 22′, analyzes the signal value and converts the signal value into a detection result, and provides whether pathogen infection exists or not, or whether pathogen infection exists or not is given out by observing the line color developing intensity of the test paper 3′ in the area of the observation window 22′ in combination with a result interpretation standard.

The reagent cap 4′ serves as a sample pre-processing device or a sample processing device, and processed sample types include not only swab samples, but also samples such as feces, blood and urine. Furthermore, the auxiliary test paper detection device in the present application can be used for non-quantitative detection and for quantitative detection.

### Embodiment 2

As shown in FIG. 5 to FIG. 7, the present disclosure provides an auxiliary test paper detection device. The auxiliary test paper detection device includes a reagent cap 4′ and a test paper cassette. The reagent cap 4′ is used for containing a detection reagent. Test paper 3′ is fixed in the test paper cassette, a sample absorption section of the test paper 3′ extends out from one end of the test paper cassette and is exposed, an observation window 22′ is formed in the test paper cassette, and a result display area of the test paper 3′ corresponds to the observation window 22′. A connecting structure is connected between the test paper cassette and the reagent cap 4′, and when the test paper cassette and the reagent cap 4′ are connected through the connecting structure, a closed space is formed in an inner cavity of the reagent cap 4′, and the sample absorption section of the test paper 3′ extends into the inner cavity of the reagent cap 4′.

The test paper cassette includes an upper cover 2′ and a lower base 1′ which are in snap fit with each other. The upper cover 2′ and the lower base 1′ are both of strip structures, a gap for accommodating test paper 3′ is formed between the upper cover 2′ and the lower base 1′, and the test paper 3′ is fixed through the snap fit cooperation of the upper cover 2′ and the lower base 1′. An observation window 22′ is formed in the upper cover 2′, a result display area of the test paper 3′ corresponds to the observation window 22′, and the sample absorption section of the test paper 3′ extends out of the length end of the test paper cassette.

A positioning hole 6′ is formed in the upper cover 2′, the positioning hole 6′ is formed in the end, away from the sample absorption section of the test paper 3′, of the test paper cassette, a positioning convex block 61′ is integrally formed on the lower base 1′ and located at the end, away from the sample absorption section of the test paper 3′, of the test paper cassette. The positioning hole 6′ and the positioning convex block 61′ are arranged correspondingly. When the upper cover 2′ and the lower base 1′ are in snap fit, the positioning convex block 61′ is embedded into the positioning hole 6′ and is in interference fit with the positioning hole 6′, so that the upper cover 2′ and the lower base 1′ are tightly combined, thereby improving the mounting stability of the test paper 3′ in the test paper cassette. And the shape of the end of the test paper cassette from which the sample absorption section of the test paper 3′ extends out, is adapted to the inner wall of the reagent cap 4′.

The connecting structure includes two handles 62′. The two handles 62′ are symmetrically hinged to the lower base 1′ at two sides of the test paper cassette in the width direction respectively, and the two handles 62′ are both located on the side of the test paper cassette close to the sample absorption section of the test paper 3′. And main bodies of the two handles 62′ extend outwards along the plug-in direction of the reagent cap′ 4, and the two handles 62′ are arc-shaped.

A certain amount of liquid can be sub-packaged in the reagent cap 4′ in advance, and the reagent cap 4′ is sealed with a sealing film or sealed by a cap, so that it is guaranteed that liquid in the device does not leak before use.

As shown in FIG. 5 to FIG. 7, when detection is needed, firstly, a sealing member of the reagent cap 4′ is opened, the test paper cassette is vertically inserted into the reagent cap 4′ with the sample absorption section of the test paper 3′ downwards until the test paper cassette is not pushed. The tail end of the upper cover 2′ and the tail end of the lower base 1′ are assembled to form a insertion portion which is gradually enlarged from right to left and is basically consistent with the inner ring of the inner cavity of the reagent cap 4′ in shape, and the insertion portion and the reagent cap 4′ are in interference fit. In addition, two handles 62′ on the lower base 1′ can simultaneously perform secondary extrusion sealing on the reagent cap 4′, so that liquid in the reagent cap 4′ can be doubly guaranteed not to overflow in the reaction process, thereby avoiding biological pollution.

In addition, the arc-shaped design of the handle 62′ at the lower base 1′ conforms to manual function science and is convenient for people to grasp manually. The sample absorption section of the test paper 3′ can be immersed into the liquid in the reagent cap 4′, and the test paper 3′ is stably mounted in the test paper cassette through the cooperation of the positioning hole 6′ and the positioning convex block 61′, so that the liquid in the reagent cap 4′ is prevented from rushing to the test paper 3′ at the moment of horizontal placement. Thus, in 10-15 minutes of a chromatographic reaction, the liquid in the reagent cap 4′ can be gradually chromatographed from the sample absorption end of the test paper 3′ to the result display area of the test paper 3′ and then to the top end of the test paper 3′ until the chromatographic reaction is finished. Finally, a value is read through inserting the device into the instrument or by observing the observation window 22′ through naked eyes.

After 10-15 minutes of the reaction is finished, the instrument obtains a signal value after scanning and interpreting the area of the observation window 22′, analyzes the signal value and converts the signal value into a detection result, and gives whether pathogen infection exists or not, or whether pathogen infection exists or not is given out by observing the line color developing intensity of the test paper 3′ in the area of the observation window 22′ in combination with a result interpretation standard.

The reagent cap 4′ serves as a sample pre-processing device or a sample processing device, and processed sample types include not only swab samples, but also samples such as feces, blood and urine. Furthermore, the auxiliary test paper detection device in the present application can be used for non-quantitative detection and also can be used for quantitative detection.

### Embodiment 3

As shown in FIG. 8 to FIG. 10, the present disclosure provides an auxiliary test paper detection device. The auxiliary test paper detection device includes a reagent cap 4′ and a test paper cassette. The reagent cap 4′ is used for containing a detection reagent. Test paper 3′ is fixed in the test paper cassette, a sample absorption section of the test paper 3′ extends out from one end of the test paper cassette and is exposed, an observation window 22′ is formed in the test paper cassette. A result display area of the test paper 3′ corresponds to the observation window 22′. A connecting structure is provided between the test paper cassette and the reagent cap 4′. When the test paper cassette and the reagent cap 4′ are connected through the connecting structure, a closed space is formed in an inner cavity of the reagent cap 4′, and the sample absorption section of the test paper 3′extends into the inner cavity of the reagent cap 4′.

The test paper cassette includes an upper cover 2′ and a lower base 1′ which are in snap fit with each other. The upper cover 2′ and the lower base 1′ are both of strip structures. A gap for accommodating test paper 3′ is formed between the upper cover 2′ and the lower base 1′, and the test paper 3′ is fixed through the snap fit cooperation of the upper cover 2′ and the lower base 1′. An observation window 22′ is formed in the upper cover 2′, a result display area of the test paper 3′ corresponds to the observation window 22′, and the sample absorption section of the test paper 3′ extends out of the length end of the test paper cassette.

Positioning mounting holes 7′ are formed in the upper cover 2′, one positioning mounting hole 7′ is formed in the middle of the end, away from the sample absorption section of the test paper 3′, of the upper cover 2′, and one positioning mounting hole 7′ is formed in each of the two sides of the end, close to the sample absorption section of the test paper 3′, of the upper cover 2′. Mounting columns 71′ are integrally formed on the lower base 1′. The number and positions of the mounting columns 71′ correspond to those of the positioning mounting holes 7′ in the upper cover 2′. During mounting, the test paper 3′ is placed in a gap formed through the cooperation of the upper cover 2′ and the lower base 1′. Each mounting column 71′ is embedded into the corresponding positioning mounting hole 7′ and is in interference fit with the corresponding positioning mounting hole 7′, so that the upper cover 2′ and the lower bottom 1′ are matched to press the test paper 3′.

The connecting structure includes a insertion member 72′. The insertion member 72′ is formed through the cooperation of the ends, close to the sample absorption section of the test paper 3′, of the upper cover 2′ and the lower base 1′. The outer contour of the insertion member 72′ corresponds to the inner ring of the inner cavity of the reagent cap 4′, and the insertion member 72′ is in interference fit with the side wall of the inner cavity of the reagent cap 4′.

A certain amount of liquid can be sub-packaged in the reagent cap 4′ in advance, and the reagent cap 4′ is sealed with a sealing film or sealed by a cap, so that it is guaranteed that liquid in the device does not leak before use.

As shown in FIG. 8 to FIG. 10, when detection is needed, a sealing member of the reagent cap 4′ is firstly opened, the test paper cassette is vertically inserted into the reagent cap 4′ with the sample absorption section of the test paper 3′ downwards until the test paper cassette is not pushed. The tail end of the upper cover 2′ and the tail end of the lower base 1′ are matched to form the insertion member 72′, a size of the insertion member 72′ is gradually increased from right to left, the shape of the insertion member 72′ is basically consistent with the large-to-small inner ring of the inner cavity of the reagent cap 4′. The insertion member 72′ and the reagent cap 4′ are in interference fit, so that it is guaranteed that liquid in the reagent cap 4′ cannot overflow in the reaction process, thereby avoiding biological pollution.

The sample absorption section of the test paper 3′ can be completely immersed into the liquid in the reagent cap 4′, the test paper 3′ is tightly clamped through the cooperation of the tail ends of the upper cover 2′ and the lower base 1′, thereby preventing the liquid in the reagent cap 4′ from rushing to the test paper 3′ at the moment of horizontal placement. In 10-15 minutes of a chromatographic reaction, the liquid in the reagent cap 4′ can be gradually chromatographed from the sample absorption end of the test paper 3′ to the result display area of the test paper 3′ and then to the top end of the test paper 3′ until the chromatographic reaction is finished. Finally, a value is read by inserting the device into the instrument or by observing the observation window 22′ through naked eyes.

After 10-15 minutes of the reaction is finished, the instrument obtains a signal value after scanning and interpreting the area of the observation window 22′, analyzes the signal value and converts the signal value into a detection result and gives whether pathogen infection exists or not, or whether pathogen infection exists or not is given out by observing the line color developing intensity of the test paper 3′ in the area of the observation window 22′ in combination with a result interpretation standard.

The reagent cap 4′ serves as a sample pre-processing device or a sample processing device, and processed sample types include not only swab samples, but also samples such as feces, blood and urine. Furthermore, the auxiliary test paper detection device in the present application can be used for non-quantitative detection and also can be used for quantitative detection.

### Embodiment 4

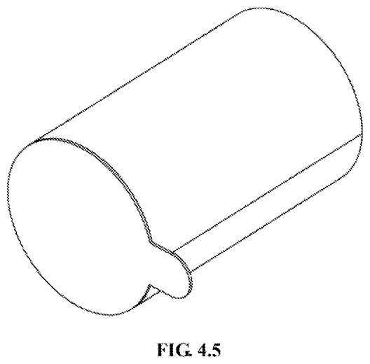

As shown in FIG. 11 to FIG. 12, the present disclosure provides an auxiliary test paper detection device. The auxiliary test paper detection device includes a reagent cap 4′ and a test paper cassette. The reagent cap 4′ is used for containing a detection reagent. Test paper 3′ is fixed in the test paper cassette, a sample absorption section of the test paper 3′ extends out from one end of the test paper cassette and is exposed. An observation window 22′ is formed in the test paper cassette. A result display area of the test paper 3′ corresponds to the observation window 22′. A connecting structure is connected between the test paper cassette and the reagent cap 4′. When the test paper cassette and the reagent cap 4′ are connected through the connecting structure, a closed space is formed in an inner cavity of the reagent cap 4′, and the sample absorption section of the test paper 3′ extends into the inner cavity of the reagent cap 4′.

The test paper cassette includes an upper cover 2′ and a lower base 1′ which are in snap fit with each other. The upper cover 2′ and the lower base 1′ are both of strip structures. A gap for accommodating test paper 3′ is formed between the upper cover 2′ and the lower base 1′. The test paper 3′ is fixed through the snap fit cooperation of the upper cover 2′ and the lower base 1′. An observation window 22′ is formed in the upper cover 2′, a result display area of the test paper 3′ corresponds to the observation window 22′. The sample absorption section of the test paper 3′ extends out from the length end of the test paper cassette.

Positioning fasteners 8 are integrally formed on the upper cover 2′ and the lower base 1′. The two positioning fasteners 8 are both located on the end of the test paper cassette away from the sample absorption section of the test paper 3′, and the two positioning fasteners 8 are in snap fit with each other. A plurality of second positioning blocking ribs 81′ are integrally formed on the upper cover 2′ and the lower base 1′. The second positioning blocking ribs 81′ are integrally formed on the upper cover 2′ and the lower base 1′ at equal intervals in the length direction of the test paper cassette.

When the upper cover 2′ and the lower base 1′ are in snap fit, the positioning fastener 8 on the upper cover 2′ is in snap fit with the positioning fastener 8 on the lower base 1′. The second positioning blocking rib 81′ on the upper cover 2′ and the positioning blocking rib 81′ on the lower base 1′ abut against the test paper 3′ tightly, so that the test paper 3′ is stably mounted in the test paper cassette

The connecting structure includes an elastic sealing member. The elastic sealing member is mounted at the end of the test paper cassette close to the sample absorption section of the test paper 3′. The elastic sealing member is mounted between the test paper 3′ and the test paper cassette and includes an inner rubber plug and an outer rubber plug 84′. The inner rubber plug includes a lower inner rubber plug 82′ and an upper inner rubber plug 83′. The shape of the end, where the sample absorption section of the test paper 3′ is located, of the test paper cassette is adapted to the outer wall of the reagent cap 4′, and the outer contour of the elastic sealing member is adapted to the inner cavity of the reagent cap 4′.

When the test paper 3′ is to be mounted in the test paper cassette, the test paper 3′ is firstly fixed to the lower inner rubber plug 82′, then the upper inner rubber plug 83′ and the lower inner rubber plug 82′ are fixed, and it is guaranteed that the test paper 3′ is fixed through interference fit between a groove of the lower inner rubber plug 82′ and a rivet of the upper inner rubber plug 83′ and interference fit between a rivet of the lower inner rubber plug 82′ and a groove of the upper inner rubber plug 83′. The tail end of the test paper 3′ is inserted into the outer rubber plug 84′, the combined inner rubber plug is embedded with the outer rubber plug 84′ by means of rubber material characteristics, the combined inner rubber plug and the outer rubber plug 84′ are tightly matched. Then the above components are arranged in the upper cover 2′ and the lower base 1′ in an upper alignment mode to be clamped, and the upper cover 2′ and the lower base 1′ are completely sealed through the snap fit cooperation of the two positioning fasteners 8.

A certain amount of liquid can be sub-packaged in the reagent cap 4′ in advance, and the reagent cap 4′ is sealed with a sealing film or sealed by a cap, so that it is guaranteed that liquid in the device does not leak before use.

As shown in FIG. 11 and FIG. 12, when detection is needed, a sealing member of the reagent cap 4′ is firstly opened, the test paper cassette is vertically inserted into the reagent cap 4′ with the sample absorption section of the test paper 3′ downwards until the test paper cassette is not pushed. It is guaranteed that the reagent bearing device is free of a liquid leakage risk through interference fit between the outer rubber plug 84′ in the test paper cassette and the large-to-small inner cavity of the reagent cap 4′ by means of rubber properties simultaneously. In addition, the tail ends of the upper cover 2′ and the lower base 1′ are enclosed to form an insertion member, the shape of the insertion member is basically consistent with the outer cavity of the reagent cap 4′, and the insertion member is in interference fit with the outer cavity of the reagent cap 4′, so that it is guaranteed that the reagent cap 4′ can be stably fixed in the test paper cassette, thereby avoiding biological pollution.

In addition, the sample absorption section of the test paper 3′ can be immersed into liquid in the reagent cap 4′. Through a press-fit between the combined inner rubber plug and the test paper 3′ as well as between the outer rubber plug and the test paper 3′, the liquid in the reagent cap 4′ is prevented from rushing to the test paper 3′ at the moment of horizontal placement. The liquid in the reagent cap 4′ can be gradually chromatographed from the sample absorption end of the test paper 3′ to the result display area of the test paper 3′ and then to the top end of the test paper 3′ until the chromatographic reaction is finished. Finally, a value is read by inserting the device into the instrument or by observing the observation window 22′ through the naked eyes.

After 10-15 minutes of the reaction is finished, the instrument obtains a signal value after scanning and interpreting the area of the observation window 22′, analyzes the signal value and converts the signal value into a detection result, and gives whether pathogen infection exists or not, or whether pathogen infection exists or not is given out by observing the line color developing intensity of the test paper 3′ in the area of the observation window 22′ in combination with a result interpretation standard.

The reagent cap 4′ serves as a sample pre-processing device or a sample processing device, and processed sample types include not only swab samples, but also samples such as feces, blood and urine. Furthermore, the auxiliary test paper detection device in the present application can be used for non-quantitative detection and also can be used for quantitative detection.

Working Principle:

During use, through the cooperation of the upper cover 2′ and the lower base 1′, the test paper 3′ is stably and fixedly clamped in a mounting gap formed through the cooperation of the upper cover 2′ and the lower base 1′, the sample absorption section of the test paper 3′ extends out from one end of the test paper cassette and is exposed. The result display area of the test paper 3′ corresponds to the observation window 22′ of the test paper cassette. A certain amount of liquid is prepackaged in the reagent cap 4′ in advance; during detection, the sealing member of the reagent cap 4′ is firstly opened, and then the test paper cassette is vertically inserted into the reagent cap 4′ with the sample absorption section of the test paper 3′ downwards until the test paper cassette is not pushed. After 10-15 minutes of the reaction is finished, the instrument obtains a signal value after scanning and interpreting the area of the observation window 22′, analyzes the signal value and converts the signal value into a detection result, and gives give whether pathogen infection exists or not, or whether pathogen infection exists or not is given out by observing the line color developing intensity of the test paper 3′ in the area of the observation window 22′ in combination with a result interpretation standard.

### Embodiment 5

FIG. 11 and FIG. 1.2 describe a device in the present disclosure. An auxiliary sample collection and detection device includes a test paper cassette and a reagent bearing device 4.

The assembled test paper cassette includes a lower base 1, an upper cover 2, a test paper 3 and a clamping sleeve 5. An accommodating groove for fixing test paper is formed in the lower base 1, the test paper 3 is fixed in the test paper accommodating groove of the lower base 1 in an upper alignment mode. The test paper is clamped and fixed through the positioning blocking ribs to be prevented from sliding inwards and outwards. When the upper cover 2 and the lower base 1 are in snap fit, the first positioning fastener 1.1 at the end of the lower base is in interference fit with the second positioning fastener 2.1 at the end of the upper cover, so that the upper cover 2 and the lower base 1 are completely sealed. The clamping sleeve 5 is clamped at the convex parts of the upper cover 2 and the lower base 1 from bottom to top until the clamping sleeve 5 is not pushed. An upper cover tail end fastener 2.3 and a lower base tail end fastener 1.3 are enclosed to form a fastener which is basically consistent with the inner ring of the clamping sleeve in shape. When the clamping sleeve is sleeved to a preset station, the inner ring of the clamping sleeve 5 abuts against and is fixed with the upper cover tail end fastener 2.3 and the lower base tail end fastener 1.3, so as to guarantee for a second time that the upper cover 2 and the lower base 1 are completely sealed.

A certain amount of liquid can be sub-packaged in the reagent bearing device 4 in advance, and the reagent bearing device is thermally sealed with a sealing film or sealed by a cap, so that it is guaranteed that liquid in the device does not leak before use.

In the embodiment, the sealing member of the reagent bearing device 4 is firstly opened, the test paper cassette is vertically inserted into the reagent bearing device 4 with the sample absorption section downwards until the test paper cassette is not pushed. The large-to-small structure of the inner ring of the reagent bearing device 4 is in interference fit with the small-to-large structure of the middle section 5.2 of the inner ring of the clamping sleeve. Therefore, when the reagent bearing device 4 is horizontally placed, liquid in the reagent bearing device 4 cannot leak into the clamping sleeve 5. Furthermore, the sample absorption section of the test paper 3 can be immersed into liquid of the reagent bearing device 4. The test paper 3 can be firmly fixed under the compression action on the test paper 3 by an upper cover positioning blocking rib 2.6 and a lower base positioning blocking rib 1.6, such compressing action also can prevent the liquid in the reagent bearing device 4 from rushing to the test paper 3 instantly when the detection device is horizontally placed. In 10-15 minutes of a chromatographic reaction, the liquid can be gradually chromatographed from the sample absorption end of the test paper 3 to a reaction window 2.2 and then to the top end of the test paper 3 until the chromatographic reaction is finished; then, a value is read by inserting the test paper into an instrument or by naked eyes.

Referring to FIG. 1.2, a reaction window 2.2 and a ventilated window 2.4 are formed in the upper end of the upper cover 2′. After 10-15 minutes of the reaction is finished, the instrument obtains a signal value after scanning and interpreting the area of the reaction window 2.2, analyzes the signal value and converts the signal value into a detection result, and gives whether pathogen infection exists or not, or whether pathogen infection exists or not is given out by observing the line color developing intensity of the test paper in the area of the reaction window 2.2 in combination with a result interpretation standard.

The reagent bearing device 4 serves as a sample preprocess device or a sample processing device, and processed sample types include not only swab samples, but also samples such as feces, blood and urine.

### Embodiment 6

FIG. 2.1 and FIG. 2.2 describe a device in the present disclosure. An auxiliary sample collection and detection device includes a test paper cassette and a reagent bearing device 4.

The assembled test paper cassette includes a lower base 1, an upper cover 2 and a test paper 3. An accommodating groove for fixing test paper is formed in the lower base 1, the test paper 3 is fixed in the test paper accommodating groove of the lower base 1 in an upper alignment mode to be clamped. When the upper cover 2 and the lower base 1 are in snap fit, the first positioning fastener 1.1 at the end of the lower base is in interference fit with the second positioning fastener 2.1 at the end of the upper cover, so that the upper cover 2 and the lower base 1 are completely sealed.

A certain amount of liquid can be sub-packaged in the reagent bearing device 4 in advance, and the reagent bearing device is thermally sealed with a sealing film or sealed by a cap, so that it is guaranteed that liquid in the device does not leak before use.

In the embodiment, a sealing member of the reagent bearing device 4 is firstly opened, and the test paper cassette is vertically inserted into the reagent bearing device 4 with the sample absorption section of the test paper downwards until the test paper cassette is not pushed. An upper cover tail end 2.31 and a lower base tail end 1.31 are enclosed to form an insertion member which is gradually enlarged from right to left and is basically consistent with the inner ring of the inner cavity of the reagent bearing device 4 in shape, the insertion member and the inner ring of the inner cavity of the reagent bearing device are in interference fit. Furthermore, a lower base handle 1.7 can perform secondary extrusion sealing on the reagent bearing device 4 at the same time, which can guarantee that the liquid in the reagent bearing device 4 cannot overflow in the reaction process, thereby avoiding the biological pollution. In addition, the arc-shaped design of the lower base handle 1.7 conforms to manual function science and is convenient for people to grasp manually. In addition, the sample absorption section of the test paper 3 can be immersed into liquid of the reagent bearing device 4, through the press fit between the lower base tail end 1.31 and the test paper 3 as well as the press-fit between the second positioning fastener 2.1 at the end of the upper cover and the test paper 3, the liquid in the reagent bearing device 4 is prevented from rushing to the test paper 3 instantly when the detection device is horizontally placed. In 10-15 minutes of a chromatographic reaction, the liquid can be gradually chromatographed from the sample absorption end of the test paper 3 to a reaction window 2.2 and then to the top end of the test paper 3 until the chromatographic reaction is finished. A value is read by inserting the test paper into an instrument or by naked eyes.

Referring to FIG. 2.2, a reaction window 2.2 is formed in the upper end of the upper cover 2. After 10-15 minutes of the reaction is finished, the instrument obtains a signal value after scanning and interpreting the area of the reaction window 2.2, analyzes the signal value and converts the signal value into a detection result, and gives whether pathogen infection exists or not, or whether pathogen infection exists or not is given out by observing the line color developing intensity of the test paper in the area of the reaction window 2.2 through naked eyes in combination with a result interpretation standard.

The reagent bearing device 4 serves as a sample pre-processing device or a sample processing device, and processed sample types include not only swab samples, but also samples such as feces, blood and urine.

### Embodiment 7

FIG. 3.1 and FIG. 3.2 describe a device in the present disclosure. An auxiliary sample collection and detection device includes a test paper cassette and a reagent bearing device 4.

The assembled test paper cassette includes a lower base 1, an upper cover 2 and a test paper 3. An accommodating groove for fixing test paper is formed in the lower base 1, the test paper 3 is fixed in the test paper accommodating groove of the lower base 1 in an upper alignment mode to be clamped. When the upper cover 2 and the lower base 1 are in snap fit, the first positioning fastener 1.1 at the end of the lower base is in interference fit with the second positioning fastener 2.1 at the end of the upper cover, and meanwhile, the first positioning fastener 1.1 at the tail end of the lower base and the second positioning fastener 2.1 at the tail end of the upper cover are also in interference fit, so that the upper cover 2 and the lower base 1 are completely sealed based on the interference fit between the top portion and the tail portion of the cover.

A certain amount of liquid can be sub-packaged in the reagent bearing device 4 in advance, and the reagent bearing device 4 is sealed with a sealing film or sealed by a cap, so that it is guaranteed that liquid in the device does not leak before use.

In the embodiment, a sealing member of the reagent bearing device 4 is firstly opened, and the test paper cassette is vertically inserted into the reagent bearing device 4 with the sample absorption section of the test paper downwards until the test paper cassette is not pushed; an upper cover tail end 2.31 and a lower bottom tail end 1.31 are enclosed to form an insertion member which is gradually enlarged from right to left and is basically consistent with the large-to-small inner ring of the inner cavity of the reagent bearing device 4 in shape, the insertion member and the inner ring of the inner cavity of the reagent bearing device are in interference fit, so that liquid in the bearing device 4 can be guaranteed not to overflow in the reaction process, thereby avoiding biological pollution. In addition, the sample absorption section of the test paper 3 can be completely immersed into liquid of the reagent bearing device 4, through the press-fit cooperation between the lower base tail end 1.31 and the test paper 3 as well as the press-fit cooperation between the upper cover tail end 2.31 and the test paper 3, the liquid in the reagent bearing device 4 is prevented from rushing to the test paper 3 instantly when the detection device is horizontally placed. In 10-15 minutes of a chromatographic reaction, the liquid can be gradually chromatographed from the sample absorption end of the test paper 3 to a reaction window 2.2 and then to the top end of the test paper 3 until the chromatographic reaction is finished; then, a value can be read by inserting the test paper into an instrument or by naked eyes.

Referring to FIG. 3.1, a reaction window 2.2 is formed in the upper end of the upper cover 2. After 10-15 minutes of the reaction is finished, the instrument obtains a signal value after scanning and interpreting the area of the reaction window 2.2, analyzes the signal value and converts the signal value into a detection result, and gives whether pathogen infection exists or not, or whether pathogen infection exists or not is given out by observing the line color developing intensity of the test paper in the area of the reaction window 2.2 through naked eyes, in combination with a result interpretation standard.

The reagent bearing device 4 serves as a sample pre-processing device or a sample processing device, and processed sample types include not only swab samples, but also samples such as feces, blood and urine.

### Embodiment 8

FIG. 4.1, FIG. 4.2 and FIG. 3 describe a device in the present disclosure. An auxiliary sample collection and detection device includes a test paper cassette and a reagent bearing device 4.

The test paper cassette includes a lower base 1, an upper cover 2, a test paper 3, a lower inner rubber plug 5, an upper inner rubber plug 6 and an outer rubber plug 7. An accommodating groove for fixing test paper is formed in the lower base 1, the test paper 3 is firstly fixed to the lower inner rubber plug 5, then the upper inner rubber plug 6 and the lower inner rubber plug 5 are fixed, and it is guaranteed that the test paper 3 is fixed through interference fit between a lower-inner-rubber-plug groove 5.1 and an upper-inner-rubber-plug rivet 6.2 and interference fit between a lower-inner-rubber-plug rivet 5.2 and an upper-inner-rubber-plug groove 6.1. The tail end of the test paper 3 is inserted into the outer rubber plug 7, the combined inner rubber plug is embedded with the outer rubber plug 7 by means of rubber material characteristics, the combined inner rubber plug and the outer rubber plug 7 are tightly matched. The above components are arranged in the lower base 1 in an upper alignment mode to be clamped, the upper cover 2 and the lower base 1 are sealed. At the upper end of the detection device, the first positioning fastener 1.1 at the end part of the lower base is in interference fit with the second positioning fastener 2.1 at the end part of the upper cover. At the tail end of the detection device, the two first positioning fasteners 1.1 at the tail portion of the lower base are in interference fit with the second positioning fastener 2.1 at the tail portion of the upper cover, which can guarantee that the upper cover 2 and the lower base 1 can be completely sealed.

A certain amount of liquid can be sub-packaged in the reagent bearing device 4 in advance, and the reagent bearing device is sealed thermally with a sealing film or sealed by a cap, so that it is guaranteed that liquid in the device does not leak before use.

In the embodiment, a sealing member of the reagent bearing device 4 is firstly opened, and the test paper cassette is vertically inserted into the reagent bearing device 4 with the sample absorption section of the test paper downwards until the test paper cassette is not pushed. It is guaranteed that the reagent bearing device is free of a liquid leakage risk through interference fit between the outer rubber plug 7 in the test paper cassette and the large-to-small inner cavity of the reagent bearing device 4 by means of rubber properties simultaneously. The tail end of the upper cover 2 and the tail end of the lower base 1 are enclosed to form an insertion member which is basically consistent with the outer cavity of the reagent bearing device in shape, the insertion member and the outer cavity of the reagent bearing device are in interference fit, so that the reagent bearing device can be stably fixed in the test paper cassette, thereby avoiding biological pollution. In addition, the sample absorption section of the test paper 3 can be immersed into liquid of the reagent bearing device 4, through the press fit between the combined inner rubber plug and the test paper 3 as well as that between the outer rubber plug 7 and the test paper 3, the liquid in the reagent bearing device 4 is prevented from rushing to the test paper 3 instantly when the detection device is horizontally placed. In 10-15 minutes of a chromatographic reaction, the liquid can be gradually chromatographed from the sample absorption end of the test paper 3 to a reaction window 2.2 and then to the top end of the test paper 3 until the chromatographic reaction is finished. A value is read by inserting the test paper into an instrument or by naked eyes.

Referring to FIG. 4.2, a reaction window 2.2 is formed in the upper end of the upper cover 2. After 10-15 minutes of the reaction is finished, the instrument obtains a signal value after scanning and interpreting the area of the reaction window 2.2, analyzes the signal value and converts the signal value into a detection result, and gives whether pathogen infection exists or not, or whether pathogen infection exists or not is given out by observing the line color developing intensity of the test paper in the area of the reaction window 2.2 through naked eyes, in combination with a result interpretation standard.

The reagent bearing device 4 serves as a sample pre-processing device or a sample processing device, and processed sample types include not only swab samples, but also samples such as feces, blood and urine.

Several examples are used for illustration of the principles and implementation methods of the present disclosure. The description of the embodiments is used to help illustrate the method and its core principles of the present disclosure. In addition, those skilled in the art can make various modifications in terms of specific embodiments and scope of application in accordance with the teachings of the present disclosure. In conclusion, the content of this specification shall not be construed as a limitation to the present disclosure.

1. An auxiliary test paper detection device, comprising a reagent cap and a test paper cassette, wherein the reagent cap is configured for containing a detection reagent, a sealing member is arranged at an opening of the reagent cap, and the sealing member is opened or removed under action of external force;
test paper is arranged in the test paper cassette, a sample absorption section of the test paper is exposed, an observation window is formed in the test paper cassette, and a result display area of the test paper corresponds to the observation window; and
a connecting structure for connecting the test paper cassette and the reagent cap is provided; when the test paper cassette and the reagent cap are connected through the connecting structure, a closed space is formed by an inner cavity of the reagent cap, and the sample absorption section of the test paper extends into the inner cavity of the reagent cap.

2. The auxiliary test paper detection device according to claim 1, wherein the test paper cassette comprises an upper cover and a lower base which are in snap fit with each other, a gap for accommodating the test paper is formed between the upper cover and the lower base, and the upper cover and the lower base each are provided with positioning fasteners and first positioning blocking ribs; and
when the upper cover and the lower base are in snap fit, the positioning fasteners on the upper cover and the lower base are in snap fit with each other to form positioning lock, and the first positioning blocking ribs on the upper cover and the lower base cooperatively and tightly abut against the test paper.

3. The auxiliary test paper detection device according to claim 2, wherein the connecting structure comprises a connecting clamping sleeve and tail end fasteners, an inner periphery of an inner ring of the connecting clamping sleeve is fixedly sleeved on the test paper cassette, and an outer periphery of the inner ring of the connecting clamping sleeve is sleeved with the reagent cap and in interference fit with the reagent cap;
the tail end fasteners are elastic, and the tail end fasteners are respectively arranged on the upper cover and the lower bottom at an end of the test paper cassette, fixedly sleeved with the connecting clamping sleeve; and
when the connecting clamping sleeve is sleeved to a predetermined station, the inner ring of the connecting clamping sleeve is in snap fit with the tail end fasteners on the upper cover and the lower base.

4. The auxiliary test paper detection device according to claim 1, wherein the test paper cassette comprises an upper cover and a lower base, a positioning hole is formed in the upper cover, a positioning convex block is arranged on the lower base, the positioning convex block is embedded in the positioning hole and in interference fit with the positioning holes; a gap for accommodating the test paper is formed between the upper cover and the lower base; and an end of the test paper cassette, from which the sample absorption section of the test paper extends out, has a shape adapted to an inner wall of the reagent cap.

5. The auxiliary test paper detection device according to claim 4, wherein the connecting structure comprises two handles, the two handles are respectively and movably arranged at two sides of the end of the test paper cassette, from which the sample absorption section of the test paper extends out, and main bodies of the two handles extend outwards in an insertion direction of the reagent cap.

6. The auxiliary test paper detection device according to claim 1, wherein the test paper cassette comprises an upper cover and a lower base which are in snap fit with each other, a gap for accommodating the test paper is formed between the upper cover and the lower base, positioning mounting holes are formed in the upper cover, and mounting columns are arranged on the lower base; and
when the upper cover and the lower base are in snap fit with each other, the mounting columns are inserted into the positioning mounting holes and in interference fit with the positioning mounting holes.

7. The auxiliary test paper detection device according to claim 6, wherein the connecting structure comprises an insertion member, an outer contour of the insertion member corresponds to a side wall of an inner cavity of the reagent cap, and the insertion member is in interference fit with the side wall of the inner cavity of the reagent cap.

8. The auxiliary test paper detection device according to claim 1, wherein the test paper cassette comprises an upper cover and a lower base which are in snap fit with each other, a gap for accommodating the test paper is formed between the upper cover and the lower base, positioning fasteners are arranged on the upper cover and the lower base, and second positioning blocking ribs are arranged on the upper cover and the lower base; and
when the upper cover is in snap fit with the lower base, the positioning fasteners on the upper cover and the lower base are in snap fit together and form a positioning lock, and the second positioning blocking ribs on the upper cover and the lower base cooperatively and tightly abut against the test paper.

9. The auxiliary test paper detection device according to claim 8, wherein the connecting structure comprises an elastic sealing member, the elastic sealing member is arranged between the test paper and the test paper cassette, and the elastic sealing member is located at an end of the test pater cassette where the sample absorption section of the test paper is located; and
the end of the test pater cassette where the sample absorption section of the test paper is located has a shape adapted to an outer wall of the reagent cap, and an outer contour of the elastic sealing member is adapted to the inner cavity of the reagent cap.

10. The auxiliary test paper detection device according to claim 8, wherein the elastic sealing member comprises inner rubber plugs and an outer rubber plug, the inner rubber plugs comprise a pair of split rubber plugs which are connected with each other in an insertion manner, the inner rubber plugs are sleeved on the test paper, one end of the outer rubber plug is sleeved on the inner rubber plugs to form secondary sealing, and another end of the outer rubber plug is inserted into the inner cavity of the reagent cap to form sealing.

11. An auxiliary detection device configured for auxiliary detection of a test paper bearing sample and display of a detection result, comprising a reagent bearing device and a test paper cassette, wherein
the reagent bearing device is a container provided with a sealing member at an opening of the container and configured for containing a certain amount of detection reagent and bearing sample solution during detection, and the sealing member is opened or removed under action of external force;
the test paper cassette is configured for positioning and assembling test paper and clamping the reagent bearing device, when the test paper and the test paper cassette are assembled, a sample absorption section of the test paper is exposed outside the test paper cassette, and a result display area of the test paper is arranged in the test paper cassette and just corresponds to a window of the test paper cassette; and when the test paper cassette clamps the reagent bearing device, the sample absorption section of the test paper is immersed into the sample solution of the reagent bearing device to implement sample bearing.

12. The auxiliary detection device according to claim 11, wherein the test paper cassette comprises an upper cover and a lower base which are in snap fit with each other, at least the result display area of the test paper is arranged between the upper cover and the lower base which are in snap fit together, and a display window is formed in the upper cover or the lower base.

13. The auxiliary detection device according to claim 12, further comprising a clamping sleeve, wherein an inner periphery of an inner ring of the clamping sleeve is sleeved on tail portions of the upper cover and the lower base, and an outer periphery of the inner ring of the clamping sleeve is sleeved with the reagent bearing device.

14. The auxiliary detection device according to claim 13, wherein working surfaces of the upper cover and the lower base are respectively and sequentially provided with end positioning fasteners, test paper accommodating grooves, positioning blocking ribs and tail end fasteners;
when the upper cover and the lower base are in snap fit together, the end positioning fasteners of the upper cover and the lower base are in snap fit with each other to form positioning lock;
the test paper accommodating grooves of the upper cover and the lower base are matched with each other to form an accommodating space for placing the test paper;
the positioning blocking ribs of the upper cover and the lower base are matched with each other to form a limiting member for preventing the test paper from sliding into and out of the test paper cassette; and
tail end fasteners of the upper cover and the lower base are assembled together to form a fastener which has a shape adapted to the inner ring of the clamping sleeve, and when the clamping sleeve is sleeved to a predetermined station, the inner ring of the clamping sleeve abuts against and is fixed with the tail end fasteners on the upper cover and on the lower base.

15. The auxiliary detection device according to claim 11, wherein a large-to-small structure of an inner ring of the reagent bearing device is in interference fit with a small-to-large structure of the inner ring of the clamping sleeve, to allow liquid in the reagent bearing device to not leak.

16. An auxiliary detection device configured for auxiliary detection of a test paper bearing sample and display of a detection result, comprising a reagent bearing device and a test paper cassette, wherein
the reagent bearing device is a container provided with a sealing member at an opening of the container and configured for containing a certain amount of detection reagent and bearing sample solution during detection, and the sealing member is opened or removed under action of external force;
the test paper cassette is configured for positioning and assembling test paper and being sleeved on the reagent bearing device, when the test paper and the test paper cassette are assembled, a sample absorption section of the test paper is exposed outside the test paper cassette, a tail portion of the test paper cassette wraps the test paper to form local sealing, and a result display area of the test paper is arranged in the test paper cassette and just corresponds to a window of the test paper cassette; and when the test paper cassette is inserted into the reagent bearing device, the tail portion of the test paper cassette and the inner cavity of the reagent bearing device form a closed space, and the sample absorption section of the test paper is immersed into the sample solution of the reagent bearing device to implement sample bearing.

17. The auxiliary detection device according to claim 16, wherein the test paper cassette comprises a cover for clamping test paper, the sample absorption section of the test paper extends out from a tail portion of the cover, the tail portion of the cover has a shape adapted to the inner cavity of the reagent bearing device, a pair of handles are arranged at two sides of the tail portion of the cover, and when the reagent bearing device with the sealing member removed is inserted into the tail portion of the cover, the pair of handles just abut against two side walls of the reagent bearing device.

18. The auxiliary detection device according to claim 17, wherein the handles are arranged at a tail portion of the lower base, one end of each handle is connected with a side wall of the lower base to form one piece, and main bodies of the handles extend outwards along an insertion direction of the reagent bearing device.

19. The auxiliary detection device according to claim 18, wherein an arc-shaped groove for fingers to grasp is formed in an outer side of each handle.

20. An auxiliary detection device configured for auxiliary detection of a test paper bearing sample and display of a detection result, comprising a test paper cassette and a reagent bearing device, wherein the test paper cassette comprises a cover for clamping test paper, a sample absorption section of the test paper extends out from a tail portion of the cover, and the tail portion of the cover has a shape adapted to an inner cavity of the reagent bearing device; and the reagent bearing device is a container provided with a sealing member at an opening of the container and configured for containing a certain amount of detection reagent and bearing sample solution during detection, and the sealing member is opened or removed under action of external force.

21. The auxiliary detection device according to claim 20, wherein the cover is configured for positioning and assembling the test paper and being sleeved on the reagent bearing device, when the test paper and the cover are assembled, the sample absorption section of the test paper is exposed outside the test paper cassette, a tail portion of the test paper cassette wraps the test paper to form local partition, and a result display area of the test paper is arranged in the test paper cassette and just corresponds to a window of the test paper cassette; and when the test paper cassette is inserted into the reagent bearing device, the tail portion of the test paper cassette and the inner cavity of the reagent bearing device form a closed space, and the sample absorption section of the test paper is immersed into the sample solution of the reagent bearing device to implement sample bearing.

22. The auxiliary detection device according to claim 21, wherein contacting surfaces of an upper cover and a lower base are respectively and sequentially provided with end positioning fasteners, test paper accommodating grooves, tail positioning fasteners and tail end insertion heads;
when the upper cover and the lower base are in snap fit, the positioning fasteners of the upper cover and the lower base are in snap fit with each other to form positioning lock;
the test paper accommodating grooves of the upper cover and the lower base are matched with each other to form an accommodating space for placing the test paper; and
tail ends of the upper cover and the lower base are assembled together to form an insertion member which has a shape adapted to the inner ring of the inner cavity of the reagent bearing device, and the insertion member is in interference fit with the inner cavity of the reagent bearing device.

23. The auxiliary detection device according to claim 22, wherein the test paper accommodating groove of the lower base is internally provided with a plurality of supporting columns used for supporting the test paper.

24. An auxiliary detection device configured for auxiliary detection of a test paper bearing sample and display of a detection result, comprising a test paper cassette and a reagent bearing device, wherein the test paper cassette comprises a cover for clamping test paper, a sample absorption section of the test paper is erected in a space in a tail portion of the cover, a sealing member is filled between the tail portion of the cover and the test paper, the tail portion of the cover has a shape adapted to an outer cavity of the reagent bearing device, and an outer contour of the sealing member is adapted to an inner cavity of the reagent bearing device; and the reagent bearing device is a container provided with a sealing member at an opening of the container and configured for containing a certain amount of detection reagent and bearing sample solution during detection, and the sealing member is opened or removed under action of external force.

25. The auxiliary detection device according to claim 24, wherein the cover is used for positioning and assembling the test paper and being sleeved on the reagent bearing device, when the test paper and the cover are assembled, the sample absorption section of the test paper is erected in a space in a tail portion of the test paper cassette; the test paper is wrapped with the sealing member, a local partition is formed between the tail portion of the test paper cassette and the test paper, and a result display area of the test paper is arranged in the test paper cassette and just corresponds to a window of the test paper cassette; when the test paper cassette is inserted into the reagent bearing device, the sealing member and the inner cavity of the reagent bearing device form a closed space, and the sample absorption section of the test paper is immersed into sample solution of the reagent bearing device to complete sample bearing work.

26. The auxiliary detection device according to claim 24, wherein the sealing member comprises inner rubber plugs and an outer rubber plug, the inner rubber plugs comprise a pair of split rubber plugs which are connected with each other in a insertion manner, the pair of inner rubber plugs are sleeved on a predetermined position of the test paper, the inner rubber plugs which are connected are inserted into one end of the outer rubber plug to form secondary sealing, and another end of the outer rubber plug is inserted into the inner cavity of the reagent bearing device to form sealing.

27. The auxiliary detection device according to claim 24, wherein the test paper cassette comprises an upper cover and a lower base which are snap fit with each other, at least the result display area of the test paper is clamped between the upper cover and the lower base, and a display window is formed in the upper cover or the lower base.

28. The auxiliary detection device according to claim 27, wherein working surfaces of the upper cover and the lower base are respectively and sequentially provided with end positioning fasteners, test paper accommodating grooves, positioning blocking ribs and tail end fasteners;
when the upper cover and the lower base are in snap fit with each other, the end positioning fasteners of the upper cover and the lower base are in snap fit with each other to form positioning lock;
the test paper accommodating grooves of the upper cover and the lower base are matched with each other to form an accommodating space for placing the test paper; and
the positioning blocking ribs of the upper cover and the lower base are matched with each other to form a limiting member for preventing the test paper to sliding in and out of the test paper cassette.

29. The auxiliary detection device according to claim 24, wherein the sealing member is a sealing film or other covers with sealing effect.

30. The auxiliary detection device according to claim 13, wherein a large-to-small structure of an inner ring of the reagent bearing device is in interference fit with a small-to-large structure of the inner ring of the clamping sleeve, to allow liquid in the reagent bearing device to not leak.

31. The auxiliary detection device according to claim 11, wherein the sealing member is a sealing film or other covers with sealing effect.

32. The auxiliary detection device according to claim 16, wherein the sealing member is a sealing film or other covers with sealing effect.

33: The auxiliary detection device according to claim 20, wherein the test paper cassette comprises the upper cover and the lower base which are snap fit with each other, at least the result display area of the test paper is clamped between the upper cover and the lower base, and a display window is formed in the upper cover or the lower base.

34. The auxiliary detection device according to claim 20, wherein the sealing member is a sealing film or other covers with sealing effect.

35. The auxiliary detection device according to claim 26, wherein the test paper cassette comprises an upper cover and a lower base which are snap fit with each other, at least the result display area of the test paper is clamped between the upper cover and the lower base, and a display window is formed in the upper cover or the lower base.
